(19) United States
(12) Patent Application Publication
(10) Pub. No.: US 2024/0382253 A1
(43) Pub. Date: Nov. 21, 2024

(54) ELECTROSURGICAL CONNECTOR ASSEMBLY

(71) Applicant: Boston Scientific Scimed, Inc., Maple Grove, MN (US)

(72) Inventors: Prateek MATHUR, Toronto (CA); Gareth DAVIES, Toronto (CA); Owen MITCHELL, Toronto (CA); Mahban SAMIEE-ZAFARGHANDY, Etobicoke (CA); Scott ZAKAIB, Toronto (CA); Kyle KAVASERI, Orangeville (CA)

(21) Appl. No.: 18/665,021

(22) Filed: May 15, 2024

Related U.S. Application Data
(60) Provisional application No. 63/502,593, filed on May 16, 2023.

(51) Int. Cl. A61B 18/14; A61B 17/00; A61B 18/00; A61B 18/12

(52) U.S. Cl. CPC A61B 18/1492; A61B 18/1206; A61B 2017/00973; A61B 2018/00083; A61B 2018/00178; A61B 2018/1253; A61B 2018/126

(57) ABSTRACT

An electrosurgical assembly for use with an electrosurgical controller is disclosed. The electrosurgical assembly includes a guidewire having an electrically conductive mandrel extending along a shaft. The distal portion of the shaft includes an electrode electrically coupled to the mandrel, and the shaft includes an electrical insulator disposed on the mandrel and extending to a proximal end. The electrosurgical assembly also includes a connector. The connector includes a cable to be coupled to the electrosurgical controller, a housing coupled to the cable, the housing having a guidewire passageway to receive the shaft of the guidewire, and a conductive member within the housing and electrically coupled to the cable. The conductive member includes a conductive contact member. The conductive contact member is adjacent the guidewire passageway to penetrate the electrical insulator and make electrical contact with the mandrel when the shaft of the guidewire is in the guidewire passageway.

### CROSS REFERENCE TO RELATED APPLICATIONS

The present application claims the benefit of U.S. Provisional Patent Application No. 63/502,593 entitled “ELECTROSURGICAL CONNECTOR ASSEMBLY,” filed May 16, 2023, the entirety of which is incorporated herein.

### TECHNICAL FIELD

The present disclosure relates to medical devices and systems for use in percutaneous or interventional procedures including surgery. More specifically, this disclosure relates to electrosurgical connectors, assemblies, and systems that provide for cutting or puncturing of bodily tissues or sensing tissue activity with an electrode.

### BACKGROUND

Catheters are often used to provide general access into a patient's body using minimally invasive techniques. In some examples, a catheter can be used to create a channel through a region of the body. One such example is a transseptal puncture in a cardiac procedure. The left atrium is a difficult cardiac chamber to access reach percutaneously. Although the left atrium can be reached via the left ventricle and mitral valve, the catheter is manipulated through two U-turns, which can be cumbersome. the transseptal puncture is a technique of creating a small surgical passage through the atrial septum, or wall in the heart between the left and right atrium, through which a catheter can be fed. The atrial septum is punctured and dilated via tools. The transseptal puncture permits a direct route to the left atrium via the intra-atrial septum and systematic venous system. Increasing larger and complex medical devices can be passed into the right atrium. Historically, the technique was used exceptionally for mitral valvuloplasty and ablation in the left heart. Today, the increased interest in catheter ablation and its application in many other procedures has meant the transseptal puncture is a routine technique for interventional cardiologists and cardiac electrophysiologists.

Transseptal punctures are now performed with the aid of guidewires having electrodes energized with a suitable power source such as an electrically coupled power generator in a manner similar to electrosurgical devices. Typical electrosurgical devices apply an electrical potential difference or a voltage difference between an active electrode and a return electrode on a patient's grounded body in a monopolar arrangement or between an active electrode and a return electrode on the device in bipolar arrangement to deliver electrical energy to the area where tissue is to be affected. The electrosurgical devices are typically held by the surgeon and connected to the power source, such as the electrosurgical unit, via cabling.

Electrosurgical devices pass electrical energy through tissue between the electrodes to cut or puncture tissue with plasma formed on the energized electrode. Tissue that contacts the plasma experiences a rapid vaporization of cellular fluid to produce a cutting effect. Typically, cutting is performed with electrodes in the monopolar arrangement. Electrical signals can be applied to the electrodes either as a train of high frequency pulses or as a continuous signal typically in the radiofrequency (RF) range to perform the cutting or puncturing techniques. The signals can include a variable set of parameters, such as power or voltage level, waveform parameters such as frequency, pulse duration, duty cycle, and other signal parameters that may be particularly apt or preferred for a given technique to form plasma.

### SUMMARY

In an Example 1, an electrosurgical assembly for use with an electrosurgical controller, the electrosurgical assembly comprising: a guidewire having an electrically conductive mandrel extending along a shaft from a proximal end to a distal portion, the distal portion having an electrode electrically coupled to the mandrel, the shaft having an electrical insulator disposed on the mandrel and extending to a proximal end; and a connector configured to releasably couple to the guidewire, the connector comprising: a cable configured to be coupled to the electrosurgical controller, a housing coupled to the cable, the housing having a guidewire passageway configured to receive the shaft of the guidewire, and a conductive member disposed within the housing and electrically coupled to the cable, the conductive member having a conductive contact member, the conductive contact member adjacent the guidewire passageway and configured to penetrate the electrical insulator and make electrical contact with the mandrel when the shaft of the guidewire is disposed in the guidewire passageway.

In an Example 2, the electrosurgical assembly of Example 1, wherein the housing includes a clamp connector configured to releasably hold the shaft of the guidewire, the clamp connector including a support member to urge the shaft against the contact member.

In an Example 3, the electrosurgical assembly of Example 2, wherein the clamp connector includes a spring coupled to the housing, wherein the spring is configured to urge the shaft against the contact member.

In an Example 4, the electrosurgical assembly of any of Examples 2 and 3, wherein the housing further includes a release button mechanically coupled to the clamp connector, the release button transitionable from a first position to a second position with respect to the housing, wherein the transition from the first position to the second position releases guidewire from the clamp connector.

In an Example 5, the electrosurgical assembly of any of Examples 1 to 4 wherein the contact member includes one of a tapered edge configured to cut the electrical insulator and a point configured to pierce the electrical insulator and make mechanical contact with the mandrel.

In an Example 6, the electrosurgical assembly of Examples 5, wherein the contact member including the edge includes one of a straight edge and a serrated edge.

In an Example 7, the electrosurgical assembly of any of claims 5 and 6, wherein the contact member includes a first contact member and a second contact member electrically coupled to the conductive member.

In an Example 8, the electrosurgical assembly of any of Examples 1 to 7, wherein the guidewire includes a plurality of electrodes.

In an Example 9, the electrosurgical assembly of any of Examples 1 to 8, wherein the connector includes a plurality of spaced-apart conductive members, and the cable includes a plurality of lead conductors, where each conductive member is electrically coupled to a corresponding lead conductor of the plurality of lead conductors, and wherein the guidewire includes a plurality of mandrels, each mandrel is coupled to a corresponding electrode of the plurality of electrodes, wherein each conductive member is couplable to a corresponding mandrel of the plurality of mandrels.

In an Example 10, the electrosurgical assembly of any of Examples 1 to 9, further comprising a plurality of ring connectors, wherein each mandrel is electrically coupled to a corresponding ring connector, and the ring connectors are longitudinally spaced-apart at a proximal end of the shaft.

In an Example 11, the electrosurgical assembly of Example 10, wherein the conductive members are radially spaced apart.

In an Example 12, the electrosurgical assembly of any of Examples 8 to 11, wherein the plurality of electrodes includes an active electrode and a return electrode, and the guidewire member is configured to operate in a bipolar mode.

In Example 13, the electrosurgical assembly of any of Examples 1 to 12, wherein the guidewire includes proximal portion of the shaft, and the housing is configured to receive the proximal portion of the shaft.

In an Example 14, the electrosurgical assembly of any of Examples 1 to 13, wherein the guidewire passageway includes one of a single opening and a plurality of openings for the guidewire passageway.

In an Example 15, the electrosurgical assembly of any of Examples 1 to 14, wherein the electrosurgical controller is one of a radiofrequency (RF) generator and an electroanatomical mapping (EAM) controller.

In an Example 16, an electrosurgical assembly for use with an electrosurgical controller, the electrosurgical assembly comprising: a guidewire having an electrically conductive mandrel extending along a shaft from a proximal end to a distal portion, the distal portion having an electrode electrically coupled to the mandrel, the shaft having an electrical insulator disposed on the mandrel and extending to a proximal end; and a connector configured to releasably couple to the guidewire, the connector comprising: a cable configured to be coupled to the electrosurgical controller, a housing coupled to the cable, the housing having a guidewire passageway configured to receive the shaft of the guidewire, and a conductive member disposed within the housing and electrically coupled to the cable, the conductive member having a conductive contact member, the conductive contact member adjacent the guidewire passageway and configured to penetrate the electrical insulator and make electrical contact with the mandrel when the shaft of the guidewire is disposed in the guidewire passageway.

In an Example 17, the electrosurgical assembly of Example 16, wherein the housing includes a clamp connector configured to releasably hold the shaft of the guidewire, the clamp connector including a support member to urge the shaft against the contact member.

In an Example 18, the electrosurgical assembly of Example 17, wherein the contact member is a first contact member, and the support member includes a second contact member electrically coupled to the electrical connector.

In an Example 19, the electrosurgical assembly of Example 17, wherein the clamp connector includes a spring coupled to the housing, wherein the spring is configured to urge the shaft against the contact member.

In an Example 20, the electrosurgical assembly of Example 17, wherein the housing further includes a release button mechanically coupled to the clamp connector, the release button transitionable from a first position to a second position with respect to the housing, wherein the transition from the first position to the second position releases guidewire from the clamp connector.

In an Example 21, the electrosurgical assembly of Example 16 wherein the contact member includes one of a tapered edge configured to cut the electrical insulator and a point configured to pierce the electrical insulator and make mechanical contact with the mandrel.

In an Example 22, the electrosurgical assembly of Example 21, wherein the contact member including the edge includes one of a straight edge and a serrated edge.

In an Example 23, the electrosurgical assembly of claim 16, wherein the guidewire includes a plurality of electrodes.

In an Example 24, the electrosurgical assembly of Example 23, wherein the connector includes a plurality of spaced-apart conductive members, and the cable includes a plurality of lead conductors, where each conductive member is electrically coupled to a corresponding lead conductor of the plurality of lead conductors, and wherein the guidewire includes a plurality of mandrels, each mandrel is coupled to a corresponding electrode of the plurality of electrodes, wherein each conductive member is couplable to a corresponding mandrel of the plurality of mandrels.

In an Example 25, the electrosurgical assembly of Example 23, further comprising a plurality of ring connectors, wherein each mandrel is electrically coupled to a corresponding ring connector, and the ring connectors are longitudinally spaced-apart at a proximal end of the shaft.

In an Example 26, the electrosurgical assembly of Example 25, wherein the conductive members are radially spaced apart.

In an Example 27, the electrosurgical assembly of Example 16, wherein the guidewire includes proximal portion of the shaft, and the housing is configured to receive the proximal portion of the shaft.

In an Example 28, the electrosurgical assembly of Example 16, wherein the guidewire passageway includes one of a single opening and a plurality of openings for the guidewire passageway.

In an Example 29, an electrosurgical system, comprising: an electrosurgical controller; a guidewire having an electrically conductive mandrel extending along a shaft from a proximal end to a distal portion, the distal portion having an electrode electrically coupled to the mandrel, the shaft having an electrical insulator disposed on the mandrel and extending to a proximal end; and a connector configured to releasably couple to the guidewire, the connector comprising: a cable configured to be coupled to the electrosurgical controller, a housing coupled to the cable, the housing having a guidewire passageway configured to receive the shaft of the guidewire, and a conductive member disposed within the housing and electrically coupled to the cable, the conductive member having a conductive bladed member, the conductive bladed member adjacent the guidewire passageway and configured to penetrate the electrical insulator and make electrical contact with the mandrel when the shaft of the guidewire is disposed in the guidewire passageway.

In an Example 30, the electrosurgical system of Example 29, wherein the electrosurgical controller is one of a radiofrequency (RF) generator and an electroanatomical mapping (EAM) controller

In an Example 31, the electrosurgical system of Example 30, wherein the electrosurgical controller is the RF generator, and the electrode is configured to operate in one of a monopolar mode and a bipolar mode.

In an Example 32, the electrosurgical system of Example 30, wherein the electrosurgical controller is the EAM system, and the guidewire includes a plurality of electrodes on the distal portion of the shaft.

In an Example 33, an electrosurgical connector for use with a guidewire having an electrically conductive mandrel extending along a shaft from a proximal end to a distal portion, the distal portion having an electrode electrically coupled to the mandrel, the shaft having an electrical insulator disposed on the mandrel and extending to a proximal end an electrosurgical controller, the electrosurgical connector comprising: a guidewire having an electrically conductive mandrel extending along a shaft from a proximal end to a distal portion, the distal portion having an electrode electrically coupled to the mandrel, the shaft having an electrical insulator disposed on the mandrel and extending to a proximal end; and a connector configured to releasably couple to the guidewire, the connector comprising: a cable configured to be coupled to the electrosurgical controller, a housing coupled to the cable, the housing having a guidewire passageway configured to receive the shaft of the guidewire, a conductive member disposed within the housing and electrically coupled to the cable, the conductive member having a conductive contact member, the conductive contact member adjacent the guidewire passageway and configured to penetrate the electrical insulator and make electrical contact with the mandrel when the shaft of the guidewire is disposed in the guidewire passageway.

In an Example 34, the electrosurgical connector of Example 33, wherein the conductor contact member includes a plurality of conductive bladed members and wherein the housing includes a clamp connector configured to releasably hold the shaft of the guidewire, the clamp connector to urge the shaft against the plurality of bladed members.

In an Example 35, the electrosurgical connector of Example 34, wherein the clamp connector includes a spring coupled to the housing, wherein the spring is configured to urge the shaft against the bladed members.

While multiple embodiments are disclosed, still other embodiments of the present invention will become apparent to those skilled in the art from the following detailed description, which shows and describes illustrative embodiments of the invention. Accordingly, the drawings and detailed description are to be regarded as illustrative in nature and not restrictive.

### BRIEF DESCRIPTION OF THE DRAWINGS

FIG. 1 is a schematic diagram illustrating an example system for treating a patient, such as a heart or the vasculature of a patient, the example system including a controller, guidewire member, and electrical connector.

FIG. 2 is a schematic diagram illustrating an example assembly including the guidewire member couplable to the electrical connector for use with the controller of the example system of FIG. 1.

FIG. 3A-3E are schematic diagrams illustrating various embodiments of a feature the electrical connector of the example assembly of FIG. 2.

FIGS. 4A-4B are schematic diagrams illustrating an embodiment of a feature of an electrical connector in the example assembly of FIG. 2.

FIGS. 5A-5B are schematic diagrams illustrating another embodiment of a feature of an electrical connector in the example assembly of FIG. 2.

FIGS. 6A-6B are schematic diagrams illustrating another embodiment of a feature of an electrical connector in the example assembly of FIG. 2.

FIGS. 7A-7B are schematic diagrams illustrating another embodiment of a feature of an electrical connector in the example assembly of FIG. 2.

FIGS. 8A-8D are schematic diagrams illustrating example configurations of a feature of housings of the electrical connectors in the example assembly of FIG. 2.

FIGS. 9A-9H are schematic diagrams illustrating example configurations of another feature of housing of the electrical connectors in the example assembly of FIG. 2.

FIGS. 10A-10B are schematic diagrams illustrating other embodiments of a feature of an electrical connector in the example assembly of FIG. 2.

FIG. 11 is a schematic diagram of a feature of the housing of FIG. 9B.

While the invention is amenable to various modifications and alternative forms, specific embodiments have been shown by way of example in the drawings and are described in detail below. The intention, however, is not to limit the invention to the particular embodiments described. On the contrary, the invention is intended to cover all modifications, equivalents, and alternatives falling within the scope of the invention as defined by the appended claims.

### DETAILED DESCRIPTION

For purposes of promoting an understanding of the principles of the present disclosure, reference is now made to the examples illustrated in the drawings, which are described below. The illustrated examples disclosed herein are not intended to be exhaustive or to limit the disclosure to the precise form disclosed in the following detailed description. Rather, these exemplary embodiments were chosen and described so that others skilled in the art may use their teachings. It is not beyond the scope of this disclosure to have a number (e.g., all) of the features in an example used across all examples. Thus, no one figure should be interpreted as having any dependency or requirement related to any single component or combination of components illustrated therein. Additionally, various components depicted in a figure may be, in examples, integrated with various ones of the other components depicted therein (or components not illustrated), all of which are within the ambit of the present disclosure.

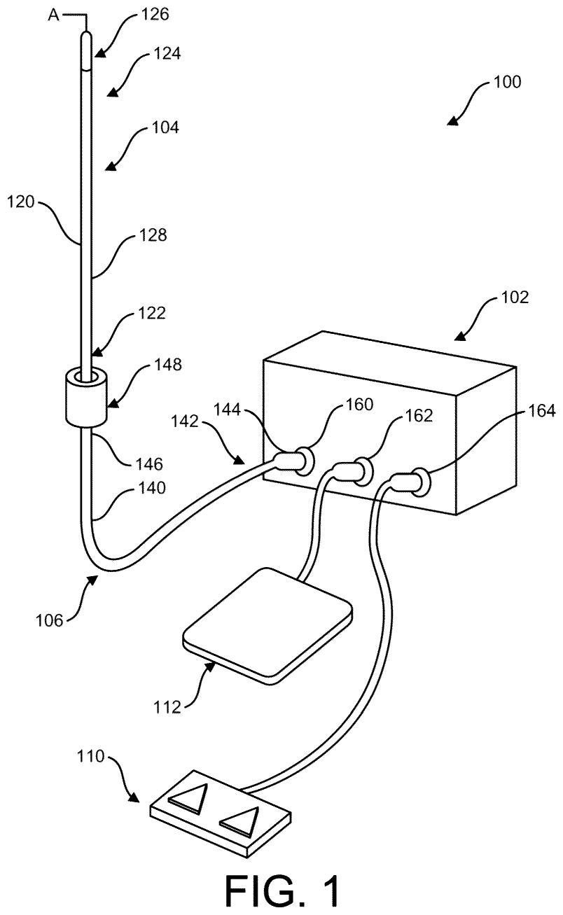

FIG. 1 illustrates an embodiment of a medical system, such as an interventional or percutaneous system 100, for treating a patient. The system 100 includes an electrosurgical controller 102 electrically coupled to a guidewire member 104 via an electrical connector 106 mechanically and electrically coupled to the controller 102 and the guidewire member 104. In some embodiments, the system includes controller switch, such as foot switch 110 and a ground pad electrode, or indifferent (dispersive) patch electrode 112 for use of the guidewire member in a monopolar configuration. In some embodiments, the guidewire member is implemented in a bipolar configuration without an indifferent patch electrode. Optionally, the system 100 can include a sheath disposed on the guidewire member 104 and a dilator.

The guidewire member 104 includes an elongate shaft 120 having a guidewire proximal portion 122 and a guidewire member distal portion 124 on an axis A. An electrode assembly 126 is disposed on the guidewire member distal portion 124. The shaft 120 includes an elongate mandrel electrically coupled to the electrode assembly 126 and extending to the guidewire member proximal portion 122. An insulate member 128 is disposed on the mandrel. In one embodiment, the insulate member 128 is covers the entire guidewire member 104 with the exception of the electrode assembly 126. For example, the insulate member initially covers the entire mandrel at the proximal portion 122. In some embodiments, the guidewire member 104 has an approximate length of 180 or 230 centimeters and an approximate diameter of 0.89 millimeters.

The electrical connector 106 includes an elongate cable 140 including a lead conductor. The cable 140 includes a cable proximal portion 142 coupled to an electrical plug 144 and a cable distal end 146 coupled to a housing 148. The lead conductor is electrically coupled to the electrical plug 144, and the electrical plug 144 is configured to be electrically and mechanically coupled to a receptable 160 on the controller 102. The housing 148 is configured to receive the insulated guidewire member proximal portion 122 and includes components to mechanically couple to the guidewire member 104 and electrically couple the lead conductor of the cable 140 to the mandrel and electrode assembly 126. In various embodiments, the cable 140 includes a number of lead conductors and the plug 144 includes a number of corresponding pins configured to electrically couple with the receptacle 160 on the controller 102. For instance, the cable 140 can include a lead conductor corresponding with each electrode in the electrode assembly 126. In such embodiments, the housing includes components configured to electrically couple the mandrels to the corresponding lead conductors in the cable 140. In one particular embodiment, the electrical connector 106 is approximately three meters in length.

The mandrel or other electrical pathways can be formed as conductive arms, wires, traces, other conductive elements, and other electrical pathways formed from electrically conductive material such as metal and in some embodiments comprise stainless steel, titanium, gold, silver, platinum, Nitinol or any other suitable material. The electrical insulator 128 formed of a biocompatible material. In some embodiments, the electrical insulator 128 is formed from a thermoplastic elastomer (TPE). For example, the TPE can be a polyether block amide (PEBA) available under the trade designation PEBAX from Arkema, S. A., of Colombes, France, or under the trade designation VESTAMID E from Evonik Industries, AG, of Essen, Germany. In some embodiments, the electrical insulator is heat shrink material such as TPE or includes polytetrafluoroethylene (PTFE).

The controller 102 provides an electrical signal to the electrode assembly 126, receives an electrical signal from the electrode assembly, or provides an electrical to an electrode in the electrode assembly 126 and receives another electrical signal from another electrode in the electrode assembly 126, via the electrical connector 106. For example, in various embodiments the controller 102 is a radiofrequency (RF) generator and the electrode assembly 126 includes an RF electrode. In various embodiments, the controller 102 is an electroanatomical mapping (EAM) console and the electrode assembly includes a sense electrode. In still other embodiment, the controller 102 is a multipurpose controller that includes multiple functions such as an RF generator and an EAM console.

In an embodiment in which the controller 102 is operated as an RF generator, the system 100 can be operated as a perforation system used in cardiac procedures or vascular procedures for advancing towards a target location with the patient's body, such as a target location in the patient's heart or location in a blood vessel and perforating the target location. For example, the guidewire member 104 can be configured as a perforation device for use in a transseptal perforation procedure. In other examples, the perforation device can be applied in intraventricular perforation procedures or venous recanalization procedures. In the example of the transseptal perforation procedure, a sheath is advanced to the right atrium of the patient's heart via a blood vessel such as the femoral vein. The perforation device and dilator are guided through the sheath to the right atrium. When the sheath is adjacent a target location in the right atrium, such as the fossa ovalis on the atrial septum, the perforation device is advanced out of the sheath and used to create a perforation at the target location. The dilator can be advanced out of the sheath to dilate the perforation.

In an embodiment in which the controller 102 is operated as an RF generator, the controller 102 further includes a receptacle 162 configured to receive a plug from the indifferent patch electrode 112 and a receptacle 164 configured to receive a plug from the foot switch 110. In various embodiments, the controller 102 can include controls such as power controls to select an amount of RF energy, a display or indicator lights and is configured to work with a suite of RF instruments or electrosurgical instruments in addition to the guidewire member 102 and electrical connector 106.

During monopolar operation of controller 102 configured as an RF generator, a first electrode, often referred to as the active electrode, is provided with the electrode assembly 126 of the guidewire member 104 while a second electrode, often referred to as the indifferent or neutral electrode, is provided in the form of the indifferent patch electrode 112 located on the patient. For example, the indifferent patch electrode 112 is typically on the back, buttocks, upper leg, or other suitable anatomical location during a procedure. The controller 102 selectively delivers a monopolar RF energy, and an electrical circuit of RF energy is formed between the active electrode and the ground pad dispersive electrode through the patient. When the active electrode of the guidewire member 104 is positioned adjacent the fossa ovalis, the controller 102 can be selectively activated, such as via foot switch 110, and the RF energy is applied to perforate the fossa ovalis.

In one example, the unit 102 provides RF energy to the active electrode as a signal having a frequency in the range of 100 KHz to 10 MHz. Typically, this energy is applied in the form of bursts of pulses. Each burst typically has a duration in the range of 10 microseconds to 1 millisecond. The individual pulses in each burst typically each have a duration of 0.1 to 10 microseconds with an interval between pulses of 0.1 to 10 microseconds. The actual pulses are often sinusoidal or square waves and bi-phasic, that is alternating positive and negative amplitudes.

In an embodiment in which the controller 102 is operated as an EAM console, the system 100 can be operated to map a patient's anatomy such as the heart. For example, the guidewire member 104 can be configured as a mapping catheter in which the electrode assembly 126 includes a sensing electrode such as a plurality of sensing electrodes configured to be used to collect electrical signals to be used to generate detailed three-dimensional geometric anatomical maps or representations of the cardiac chambers as well as electro-anatomical maps in which cardiac electrical activity of interest is superimposed on the geometric anatomical maps via an EAM system coupled to the controller 102. The guidewire member 104 can be introduced and advanced through a patient's vasculature and into the heart in a manner similar to described above. The EAM system is operable to track the location of the various components of the system 110, such as the electrode assembly 126 on guidewire member 104 such as via tracking or navigational sensors, and to generate high-fidelity three-dimensional anatomical and electro-anatomical maps of the heart, including portions of the heart such as cardiac chambers of interest or other structures of interest such as the fossa ovalis. A clinician can map the heart in the region of the fossa ovalis in order to determine a region to puncture the fossa ovalis. In one illustrative example, the EAM system can include the RHYTHMIA™ HDx mapping system marketed by Boston Scientific Corporation. For instance, in a multifunction system, sensing electrodes of the electrode assembly 126 can be electrically coupled via the electrical connector 106 to the controller 102 to map the heart when the controller 102 is configured in a mapping function. The plug 144 can include a plurality of pins, and each pin can correspond with a lead conductor in the cable 140, that associated with a mandrel in the shaft 120 of the guidewire member 104. In an embodiment of a multifunction controller, the controller 102 can be configured to receive electrical signals from the tracking or navigational sensors and the sensing electrodes of the electrode assembly 126 when the controller is configured in an EAM function, and the controller 102 can be configured to deliver RF energy to an RF electrode of the electrode assembly 126 at a target location on the heart to perforate or ablate the location when the controller 102 is configured in a puncturing function.

Often, a guidewire used in a typical system features a shaft having a PTFE insulation over a mandrel, and the mandrel is exposed from under the insulation at the distal end of the shaft. The exposed mandrel is provided to an electrical connector where the mandrel is electrically coupled to a cable. Often, there is an abrupt change in diameter at a transition between the exposed mandrel and the insulated shaft on the guidewire. The tip of a dilator can become stuck at this transition, which can make for an awkward procedure or even damage insulation or the dilator.

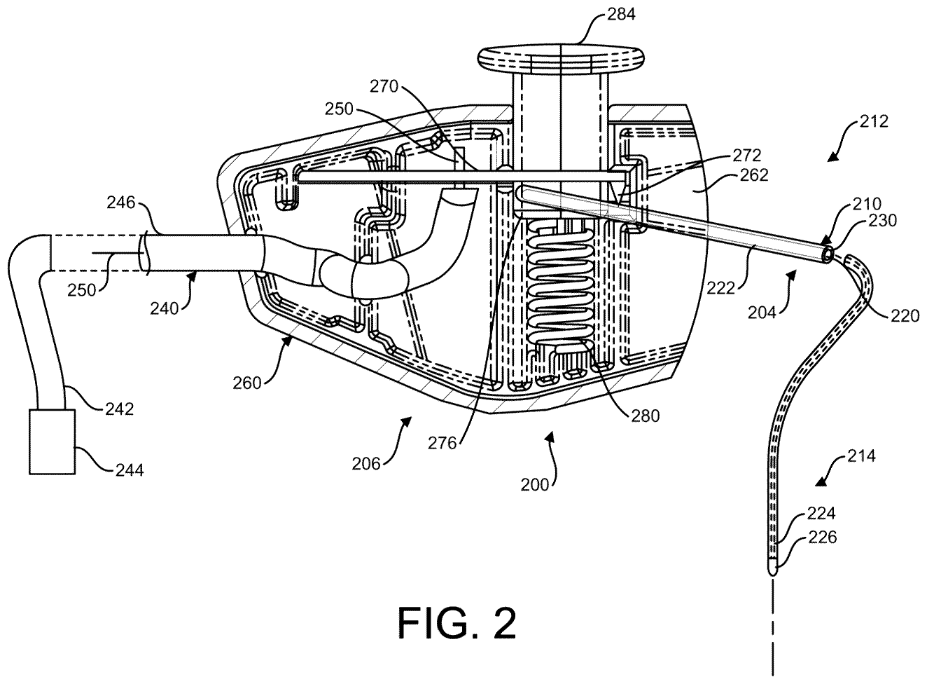

FIG. 2 illustrates an example interventional assembly 200 for use with an electrosurgical controller, such as controller 102. The assembly 200 includes a guidewire 204 and an electrical connector 206. The guidewire 204 is releasably couplable to the electrical connector 206.

The guidewire 204 includes a shaft 210 having a shaft proximal portion 212 and a shaft distal portion 214. The shaft 210 includes an electrically conductive mandrel 220 having a mandrel proximal end 222 to a mandrel distal portion 224. The shaft distal portion 214 includes an electrode 226 electrically coupled to the mandrel 220 at the mandrel distal portion 224, and the electrode 226 is exposed on the shaft 210. The shaft proximal portion 212 includes an electrical insulator 230 disposed on the mandrel 220 and covering the entire mandrel proximal end 222.

The electrical connector 206 includes an elongate cable 240 coupled to a housing 260. The cable 240 includes an elongate lead conductor 250. The cable 240 includes a cable proximal portion 242 coupled to an electrical plug 244 and a cable distal end 246 coupled to the housing 260. The lead conductor 250 is electrically coupled to the electrical plug 244, and the electrical plug 244 is configured to be electrically and mechanically coupled to the controller 102. The housing 260 is configured to be coupled to the guidewire 204. The housing 260 includes a guidewire passageway 262 configured to receive the proximal portion 212 of the shaft 210, such as the proximal end of the shaft 210 including the proximal end of the mandrel 222. A conductive member 270 is disposed within the housing 260 and electrically coupled to the lead conductor 250 of the cable 240. The conductive member 270 includes a contact member 272 adjacent to the guidewire passageway 262. the conductive member 270 and contact member 272 are constructed from a conductive material. The contact member 272 is configured to penetrate the electrical insulator 230 and make mechanical contact with the mandrel 220 when the proximal portion 212 of the shaft 210 is disposed in the guidewire passageway 262. The contact member 272 is also referred to as a bladed member 272. The conductive member 270 provides an electrical connection between the mandrel 220 and the lead conductor 250 in the guidewire passageway 262.

In various embodiments, the housing 260 further includes a clamp connector 276 configured to releasably hold the proximal portion 212 of the shaft 210 in the guidewire passageway 262. In some embodiments, the clamp connector 276 includes the bladed member 272 or is in combination with the bladed member 272. The clamp connector 276 includes a support member 278 to urge the proximal portion 212 of the shaft 210 against the bladed member 272. In various embodiments, the clamp connector 276 includes a spring 280 coupled to the housing 260, and the spring 280 is configured to yieldably urge the support member 278 against the proximal portion 212 of the shaft 210 toward the bladed member 272. In some embodiments, the clamp connector 276 includes or is in combination with a release button 284 disposed on the housing 260. The release button 284 in the illustrated embodiment is mechanically coupled to the clamp connector 276 and transitionable from a first position to a second position with respect to the housing 260. In the release button 284 is disposed in the first position when the clamp connector 276 holds the proximal portion 212 of the shaft 210, and the button 284 is moved to the second position to allow the release of the proximal portion 212 of the shaft 210 in the guidewire passageway 262.

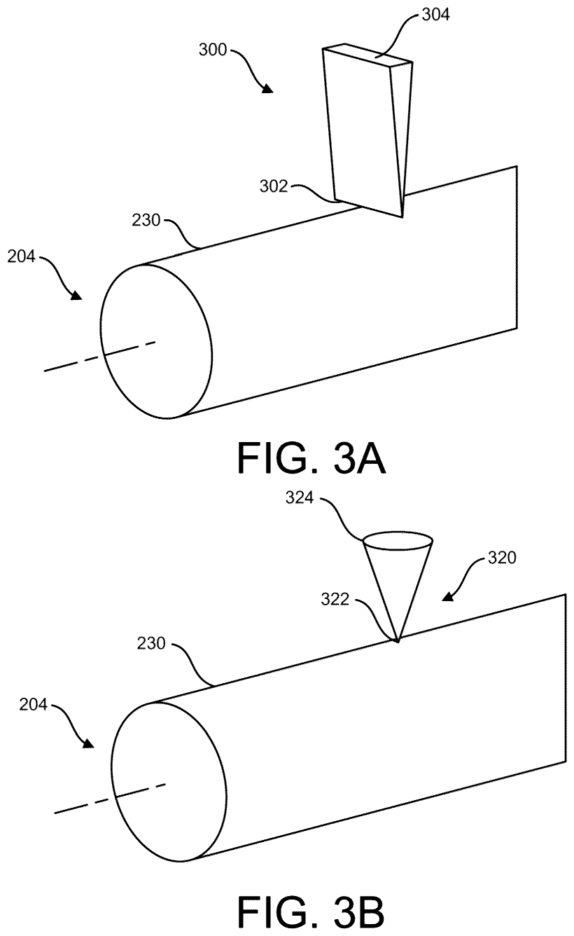

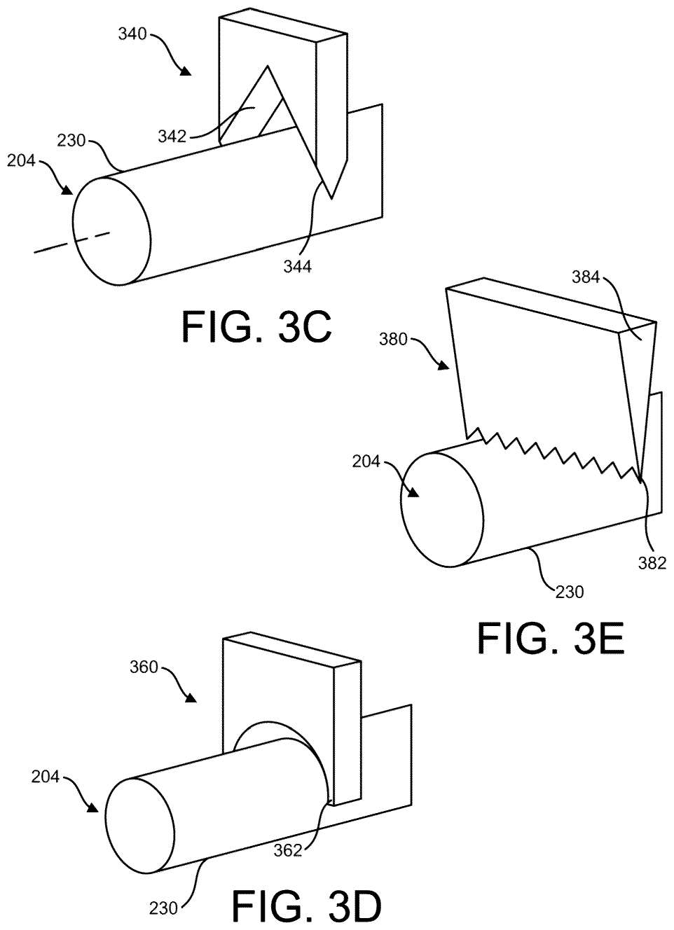

FIGS. 3A-3E illustrate various embodiments of the bladed member 272. The bladed member 272 is constructed from a conductive material and electrically coupled to the lead conductor 250 in the cable 240. The bladed member 272 is further configured to mechanically penetrate, such as cut or pierce, the electrical insulator 230 disposed on the conductive mandrel 220 covering the entire mandrel proximal end 222 of a guidewire 204 and make mechanical contact with the mandrel 220. The conductive bladed member 272 in mechanical contact with the conductive mandrel 220 forms an electrical connection between the electrode 226 on the guidewire 204 and the lead conductor 250 in the cable 240. Other embodiments than the illustrated embodiments are contemplated. For instance, the embodiments in FIGS. 3A-3E illustrate a tapered edge or sharp edge such as a razor or pin. The bladed member 272 in some embodiments does not include a tapered edge, and in some embodiments includes a uniform dimension or broadening dimension but is still sufficient to penetrate the electrical insulator 230 and contact the mandrel 220 such as to punch through or otherwise penetrate the electrical insulator 230.

FIG. 3A illustrates a first embodiment of the bladed member as bladed member 300. Bladed member 300 is configured with a sharp edge 302 opposite a proximal edge 304 that is coupled to a conductive member, which is electrically coupled to a cable. The bladed member 300 is configured similar to a razor blade in that the sharp edge 302 is configured to cut through the electrical insulator 230 and make mechanical contact with the mandrel 220, and the proximal edge 304 distributes forces applied to the bladed member 300 as the straight sharp edge 302 is pressed against the proximal portion 212 of the shaft 210. The bladed member 300 creates a straight cut in the electrical insulator 230 and is suited for making cuts on the shaft 210 longitudinally in the direction of an axis of the shaft 210 or orthogonal to the axis. For instance, the sharp edge 302 of the bladed member can be configured to be oriented longitudinally, or along the axis, of the couplable shaft 210, or orthogonal to the shaft.

FIG. 3B illustrates a second embodiment of the bladed member as bladed member 320. Bladed member 320 is configured with a pointed tip 322 opposite a proximal end 324 that is coupled to a conductive member, which is electrically coupled to a cable. The bladed member 320 is configured to pierce or puncture through the electrical insulator 230 and make mechanical contact with the mandrel 220, and the proximal edge 324 distributes forces applied to the bladed member 320 as the pointed tip 322 is pressed against the proximal portion 212 of the shaft 210. In various embodiments, the pointed tip 322 provides less damage to the electrical insulator 230 than other configurations of bladed members and can be useful if the guidewire is often removed from and reinserted into the housing 260 during a procedure.

FIG. 3C illustrates a third embodiment of the bladed member as bladed member 340. Bladed member 340 is configured as a notched blade having a plurality of straight sharp edges 342, 344 at an angle of less than 180 degrees and a proximal edge 346. Each sharp edge 342, 344 is configured similar to a razor blade in that the straight sharp edges 342, 344 are configured to cut through the electrical insulator 230 and make mechanical contact with the mandrel 220, and the proximal edge 346 distributes forces applied to the bladed member 340 as the sharp edges 342, 344 are pressed against the proximal portion 212 of the shaft 210. The bladed member 340 creates multiple points of contact with the mandrel 220 and can provide for improved stability in holding the guidewire 204 within the housing 260. For instance, the notched blade of bladed member 340 is suited for making cuts on the shaft 210 in a direction orthogonal to the axis of the shaft 210.

FIG. 3D illustrates a fourth embodiment of the bladed member as bladed member 360. Bladed member 360 is configured as a concave curve blade having a rounded sharp edge 362. Depending on the selected radius of curvature of the rounded sharp edge 362 and the diameter of the mandrel 220 at the proximal portion 212 of the shaft 210, the rounded shaft edge 362 can make increased mechanical contact over other embodiments of bladed members. The increased contact with the mandrel 220 can provide for improved stability in holding the guidewire 204 within the housing 260. The bladed member 360 is suited for making cuts on the shaft 210 in a direction orthogonal to the axis of the shaft 210.

FIG. 3E illustrates another embodiment of the bladed member as bladed member 380. Bladed member 380 is configured as a sharp edge blade 382, similar to sharp edge blade 302, but with a serrated or sawtooth edge rather than a straight edge blade as illustrate in FIG. 3A. The serrated sharp edge 382 is opposite a proximal edge 384 that is coupled to a conductive member. The bladed member 380 is configured to cut through the electrical insulator 230 and make mechanical contact with the mandrel 220, and the proximal edge 384 distributes forces applied to the bladed member 380 as the sharp edge 382 is pressed against the proximal portion 212 of the shaft 210. The bladed member 380 creates a straight cut in the electrical insulator 230 and is suited for making cuts on the shaft 210 longitudinally in the direction of an axis of the shaft 210 or orthogonal to the axis. For instance, the sharp edge 382 of the bladed member can be configured to be oriented longitudinally, or along the axis, of the couplable shaft 210, or orthogonal to the shaft 210. In some embodiments, a serrated or sawtooth blade is useful when the edge is moved laterally and along the insulator rather than simply down into it. A serrated or sawtooth edge can be applied to other configurations of the bladed member such as to a notched blade 342 or a rounded edge 362. For instance, the straight sharp edges illustrated in notched blade 340 can be substituted with a serrated edge notched blade and the rounded blade 362 illustrated with in the concave curve blade 360 can be substituted with a serrated edge rounded blade.

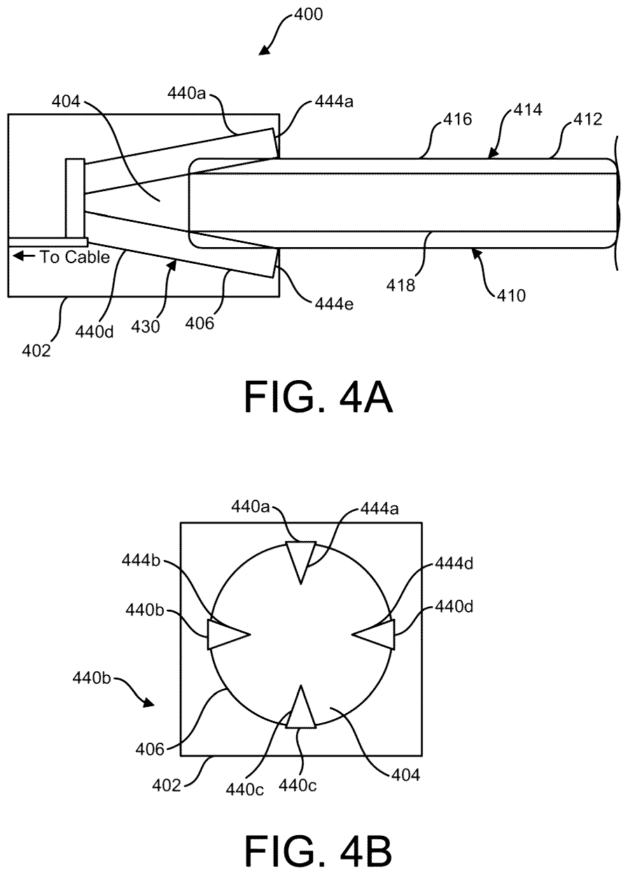

FIGS. 4A and 4B illustrate an embodiment of a feature of an electrical connector 400 for use with an interventional assembly of the electrical connector 400 couplable to a guidewire or the electrosurgical system 100. FIG. 4A is a schematic view from a side of a portion of the connector 400 illustrating a housing 402 including a guidewire passageway 404. FIG. 4B is schematic view from the front of the connector 400 illustrating the housing 402 and the guidewire passageway 404. A guidewire 410 is disposed in the guidewire passageway 404. The guidewire 410 includes a proximal portion 412 of a shaft 414. The proximal portion 412 includes an electrical insulator 416 disposed on a mandrel 418 in which the electrical insulator 416 covers the entire mandrel proximal portion 412 prior to the guidewire 410 being inserted into the guidewire passageway 404. The mandrel 418 is conductively coupled to an electrode exposed on the distal end of the guidewire 410.

The guidewire passageway 404 is defined by a conical wall 406, or walls that taper from an opening 408 in the housing 402. The housing 402 includes a clamp connector 430 disposed in the guidewire passageway 404. The clamp connector 430 is formed of an electrically conductive material and includes a plurality of conductive members 440a, 440b, 440c, 440d radially spaced around the conical wall 406. The conductive members 440 are coupled to together with a conductive extension member 442 that is mechanically and electrically coupled to the lead conductor of the cable. Each of the plurality of conductive members 440 is coupled to, such as integrally formed with in the illustrated embodiment, a corresponding bladed member 444a, 444b, 444c, 444d. The bladed members 444 in the illustrated embodiment are straight sharp edge bladed members. In another embodiment the bladed members include pointed tips. In the illustrated embodiment, the size of the guidewire passageway 404 is based on the guidewire 410 and narrows from the opening 408 of a diameter generally greater than an outside diameter of the proximal portion 412 of the guidewire 410 to a diameter generally less than the outside diameter of the proximal portion of the mandrel 418. More particularly, the radial spacing of the tips of the bladed members 444 narrow from a diameter generally greater than an outside diameter of the proximal portion 412 of the guidewire 410 at the opening 408 of the guidewire passageway 404 to a diameter generally less than the outside diameter of the proximal portion of the mandrel 418.

As the proximal portion 412 of the guidewire 410 with the intact electrical insulator 416 is inserted into the guidewire passageway 404 of the housing 402 along the direction of the longitudinal axis of the guidewire 410, the bladed members 444 come into contact with the electrical insulator 416. The size and spacing of the bladed members 444 with respect to the guidewire 410, electrical insulator 416, and mandrel 418 cause the bladed members 444 to cut through the insulator 416 and make mechanical contact with the mandrel 418 as the guidewire 410 is fed into the guidewire passageway 404. The guidewire 410 is held in place by a friction fit between the conductive members 440, which act as support members. The bladed members 444 in mechanical contact with the mandrel 418 provide an electrical connection between the mandrel 418 and the lead conductor in the cable conductively coupled to the conductive members via the clamp connector 430.

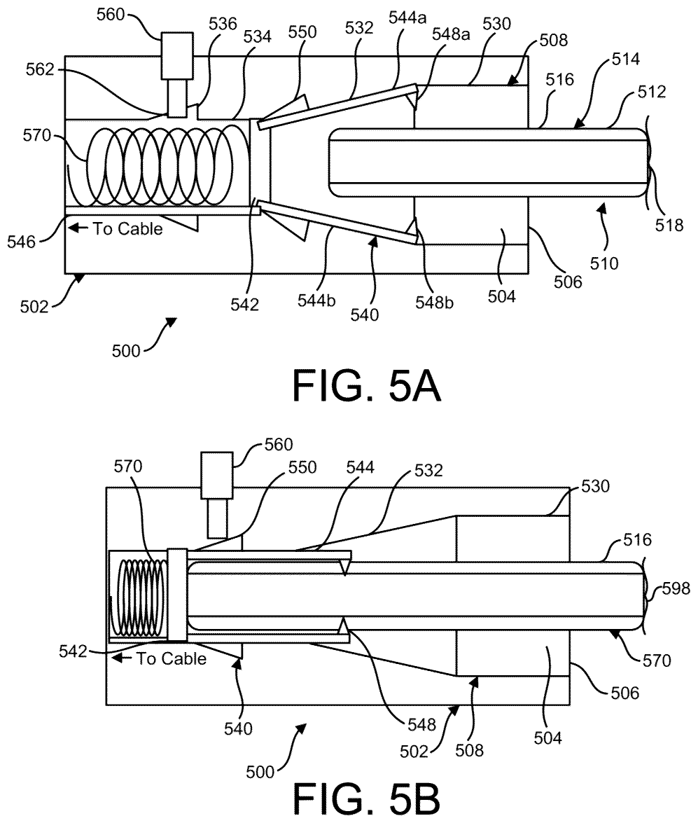

FIGS. 5A and 5B illustrate an embodiment of a feature of an axial loading electrical connector 500 for use with an interventional assembly of the electrical connector 500 couplable to a guidewire or the electrosurgical system 100. FIG. 5A is a schematic view illustrating a portion of the connector 500 having a housing 502 and guidewire passageway 504 in a released configuration with respect to a guidewire 510 in the guidewire passageway 504. FIG. 5B is a schematic view illustrating the portion of the connector 500 in a locked configuration with respect to the guidewire 510 in the guidewire passageway 504. The guidewire 510 includes a proximal portion 512 of a shaft 514. The proximal portion 512 includes an electrical insulator 516 disposed on a mandrel 518 in which the electrical insulator 516 covers the entire mandrel proximal portion 512 prior to the guidewire 510 being inserted into the guidewire passageway 504. The mandrel 518 is conductively coupled to an electrode exposed on the distal end of the guidewire 510.

The guidewire passageway 504 is defined by an opening 506 on the housing 502 and a wall 508 within the housing 502. In the illustrated embodiment, the wall 508 of the guidewire passageway 504 includes a wide cylindrical portion 530 proximate the opening 506 and a narrowing conical or taper portion 532 and a narrow cylindrical portion 534 as the guidewire passageway 504 extends into the housing 502. The narrow cylindrical portion 534 further defines a wing recess 536 having a distal straight segment proximally tapering toward the cylindrical portion 534. A clamp connector 540 is disposed in the guidewire passageway 504. In the illustrated embodiment, the clamp connector 540 is formed of an electrically conductive material and includes a base member 542 coupled to a plurality of longitudinally extending conductive members 544a, 544b radially spaced-apart in the guidewire passageway 504. The clamp connector 540 is mechanically and electrically coupled to an extension member 546 that is mechanically and electrically coupled to the lead conductor of the cable. Each of the plurality of the conductive members 544a, 544b is coupled to a corresponding bladed member 548a, 548b. The bladed members 548 extend in a radial direction of the guidewire 510. Various embodiments include at least one wing extension lock 550 on the clamp connector 540, such as wing extension lock 550 disposed on conductive member 544a. The housing 502 includes a pushbutton 560, such as a pushbutton having a first end 562 disposed in the wing recess 536. A spring 570 is disposed in the narrow cylindrical portion between the housing 502 and the clamp connector 540 urging the clamp connector 540 toward the opening 506.

In the released configuration as illustrated in FIG. 5A, the conductive members 544 are disposed against the conical portion 532 of the wall 508. The bladed members 548 extending radially inwardly from the conductive members 544 are spaced-apart from each other at a distance generally greater than the diameter of the proximal portion 512 of the guidewire 510. As the proximal portion 512 of the guidewire 510 is inserted into the guidewire passageway 506 along the direction of the longitudinal axis of the guidewire 510, the proximal tip of the guidewire presses against the clamp connector 540, such as the base member 542, compresses the spring 570, and moves the clamp connector 540 away from the opening 506 in the housing 502. The conductive members 544, tracking the shape of the tapering conical portion 532 of the wall 508, move the bladed members 548 toward the guidewire 510. Also, the wing extension lock 550 moves longitudinally toward the wing recess 536.

As the guidewire 510 is pressed deeper into the guidewire passageway 504, the electrical connector 500 transitions to the lock configuration illustrated in FIG. 5B. In the locked configuration, the spring 570 is fully compressed in the narrow cylindrical portion 534 of the guidewire passageway 504, and the wing extension lock 550 is engaged in the wing recess 536 to prevent the spring 570 from pressing the clamp connector 540 toward the opening 506. The shape of the wall 508 has urged bladed members toward the guidewire 510 such that the bladed members 548 penetrate the electrical insulator 516 and mechanically contact the mandrel 518 of the guidewire 510. The clamp connector 540 is configured such that the bladed members 548 are spaced apart at a distance about the same or closer than the diameter of the mandrel 518 in the proximal portion 512 of the shaft 514. The guidewire 510 is held in place by a friction fit between the conductive members bladed members 548, which act as support members. The bladed members 548 in mechanical contact with the mandrel 518 provide an electrical connection between the mandrel 518 and the lead conductor in the cable conductively coupled to the conductive members via the clamp connector 540.

To release the guidewire 510 from the housing 502 in the locked position, the pushbutton 560 is depressed. The first end 562 of the pushbutton disposed in the wing recess 536 deflects the wing extension lock 550 out of the wing recess 536 and into the guidewire passageway 506. The spring 570, under tension, urges the clamp connector 540 forward now with the wing extension lock 550 disengaged, and the conductive members 544 spread radially from the guidewire 510. In some embodiments, the clamp connector 540 is formed with a conductive shape memory material such as Nitinol or include springs to urge the conductive members 544 into a spread position at rest.

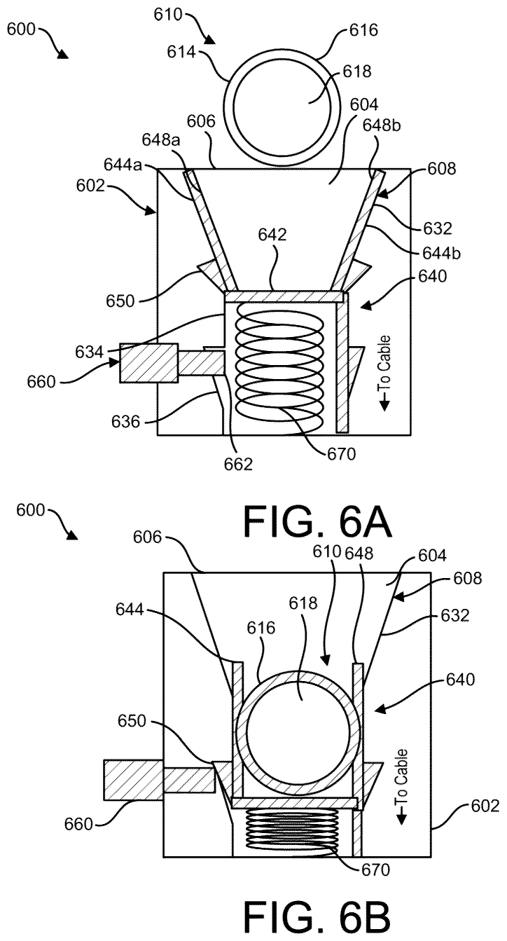

FIGS. 6A and 6B illustrate an embodiment of a feature of a side loading electrical connector 600 for use with an interventional assembly of the electrical connector 600 couplable to a guidewire or the electrosurgical system 100. FIG. 6A is a schematic view illustrating a portion of the connector 600 having a housing 602 and guidewire passageway 604 in a released configuration with respect to a guidewire 610 in the guidewire passageway 604. FIG. 6B is a schematic view illustrating the portion of the connector 600 in a locked configuration with respect to the guidewire 610 in the guidewire passageway 604. The guidewire 610 includes a shaft 614. The shaft 614 includes an electrical insulator 616 disposed on a mandrel 618 in which the electrical insulator 616 covers the entire mandrel 618 prior to the guidewire 610 being inserted into the guidewire passageway 604. The mandrel 618 is conductively coupled to an electrode exposed on the distal end of the guidewire 610. Features of axial loading connector 500 of FIGS. 5A and 5B are similar to the side loading electrical connector 600.

The guidewire passageway 604 is defined by an opening 606 on the housing 602 and a wall 608 within the housing 602. In the illustrated embodiment, the wall 608 of the guidewire passageway 604 includes a narrowing conical or taper portion 632 proximate the opening 606 and a cylindrical portion 634 as the guidewire passageway 604 extends into the housing 602. The cylindrical portion 634 further defines a wing recess 636 having a distal straight segment proximally tapering toward the cylindrical portion 634. A clamp connector 640 is disposed in the guidewire passageway 604. In the illustrated embodiment, the clamp connector 540 is formed of an electrically conductive material and includes a base member 642 coupled to a plurality of longitudinally extending conductive members 644a, 644b radially spaced-apart in the guidewire passageway 604. The clamp connector 640 is mechanically and electrically coupled to an extension member 646 that is mechanically and electrically coupled to the lead conductor of the cable. Each of the plurality of the conductive members 644a, 644b is coupled to a corresponding bladed member 648a, 648b. The bladed members 648 extend along the conductive members 644 and in a radial direction of the guidewire 610. Various embodiments include at least one wing extension lock 650 on the clamp connector 640, such as wing extension lock 650 disposed on conductive member 644a. The housing 602 includes a pushbutton 660, such as a pushbutton having a first end 662 disposed in the wing recess 636. A spring 670 is disposed in the narrow cylindrical portion between the housing 602 and the clamp connector 640 urging the clamp connector 640 toward the opening 606.

In the released configuration as illustrated in FIG. 6A, the conductive members 644 are disposed against the conical portion 632 of the wall 608. The bladed members 648 extending radially inwardly from the conductive members 644 are spaced-apart from each other at a distance generally greater than the diameter of the proximal portion 612 of the guidewire 610. As the proximal portion 612 of the guidewire 610 is inserted into the guidewire passageway 606 along the direction orthogonal to the longitudinal axis of the guidewire 610, or from the side, the side of the guidewire presses against the clamp connector 640, such as the base member 642, compresses the spring 670, and moves the clamp connector 640 away from the opening 606 in the housing 602. The conductive members 644, tracking the shape of the tapering conical portion 632 of the wall 608, move the bladed members 648 toward the guidewire 610. Also, the wing extension lock 650 moves longitudinally toward the wing recess 636.

As the guidewire 610 is pressed deeper into the guidewire passageway 604, the electrical connector 600 transitions to the lock configuration illustrated in FIG. 6B. In the locked configuration, the spring 670 is fully compressed in the cylindrical portion 634 of the guidewire passageway 604, and the wing extension lock 650 is engaged in the wing recess 636 to prevent the spring 670 from pressing the clamp connector 640 toward the opening 606. The shape of the wall 608 has urged bladed members toward the guidewire 610 such that the bladed members 648 penetrate the electrical insulator 616 and mechanically contact the mandrel 618 of the guidewire. The clamp connector 640 is configured such that the bladed members 648 are spaced apart at a distance about the same or closer than the diameter of the mandrel 618 in the proximal portion 612 of the shaft 614. The guidewire 610 is held in place by a friction fit between the conductive members bladed members 648, which act as support members. The bladed members 648 in mechanical contact with the mandrel 618 provide an electrical connection between the mandrel 618 and the lead conductor in the cable conductively coupled to the conductive members via the clamp connector 640.

To release the guidewire 610 from the housing 602 in the locked position, the pushbutton 660 is depressed. The first end 662 of the pushbutton disposed in the wing recess 636 deflects the wing extension lock 650 out of the wing recess 636 and into the guidewire passageway 606. The spring 670, under tension, urges the clamp connector 640 forward now with the wing extension lock 650 disengaged, and the conductive members 644 spread radially from the guidewire 610.

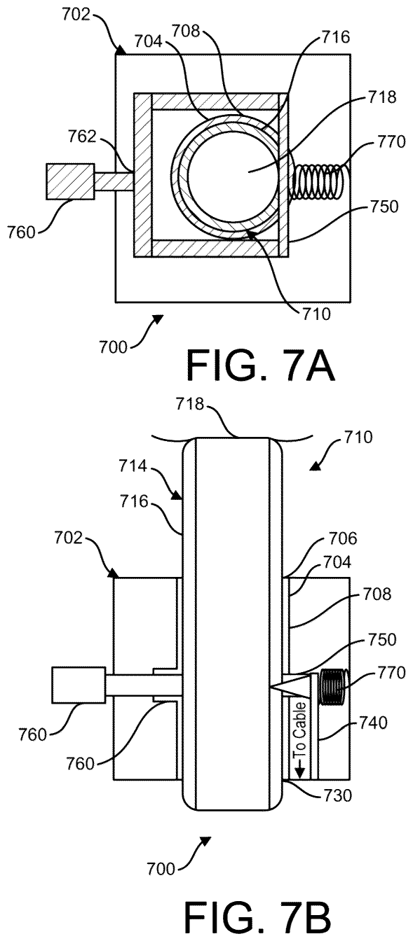

FIGS. 7A and 7B illustrate an embodiment of a feature of an electrical connector 700 for use with an interventional assembly of the electrical connector 700 couplable to a guidewire or the electrosurgical system 100. FIG. 7A is a schematic view illustrating a sectioned side view of a portion of the connector 700 having a housing 702 and guidewire passageway 704 in a locked configuration with respect to a guidewire 710 in the guidewire passageway 704. FIG. 7B is a schematic view illustrating a sectioned top view of the portion of the connector 700 in the locked configuration with respect to the guidewire 710 in the guidewire passageway 704. The guidewire 710 includes a shaft 714. The proximal portion 712 includes an electrical insulator 716 disposed on a mandrel 718 in which the electrical insulator 716 covers the entire mandrel 718 prior to the guidewire 710 being inserted into the guidewire passageway 704. The mandrel 718 is conductively coupled to an electrode exposed on the distal end of the guidewire 710.

The guidewire passageway 704 is defined by an opening 706 on the housing 702 and a wall 708 within the housing 702. In the illustrated embodiment, the guidewire 710 is received into the opening 706 of the guidewire passageway 704 axially. In the illustrated embodiment, the wall 708 of the guidewire passageway 704 defines a cylindrical passageway in the housing 702 with a second opening 730 opposite the housing 702 from the first opening 706. A conductive member 740 is disposed in the guidewire passageway 704 along a side of the wall 708. The conductive member 740 is coupled to a straight edge bladed member 750 orthogonal to the axial guidewire passageway 704, in the illustrated embodiment. The conductive member 740 is mechanically and electrically coupled to a lead conductor of the cable. The housing 702 includes a pushbutton 760, such as a pushbutton having a first end 762 coupled to the conductive member 740. A spring 770 is disposed in the housing 702 between a wall of the housing and the conductive member 740 urging the conductive member connector 740 toward an opposite side across from the guidewire passageway 704. The pushbutton 760 can be depressed to compress the spring 770 and move the conductive member 740 from the guidewire passageway 704.

To insert the guidewire 710 into the guidewire passageway 704, the pushbutton 760 is depressed and the conductive member 740 is moved out of the way of the guidewire passageway 704. Once the guidewire 710 is inserted into the guidewire passageway 704, the pushbutton 760 can be released and the spring 770 urges the guidewire 710 against the wall 708 of the guidewire passageway 704, which acts as a support member, and urges the bladed member 750 into the electrical insulator 716. The bladed member 750 penetrates the electrical insulator 716 and mechanically contacts the mandrel 718 of the guidewire 710. The guidewire 710 is held in place with respect to the connector 700 by a friction fit between the bladed member 750 and the wall 708, which act as a support member. The bladed member 750 in mechanical contact with the mandrel 718 provide an electrical connection between the mandrel 718 and the lead conductor in the cable conductively coupled to the conductive members 740. To release the guidewire 710 from the connector 700, the pushbutton 760 is depressed and the bladed member 750 is moved away from the guidewire 710 releasing the friction fit.

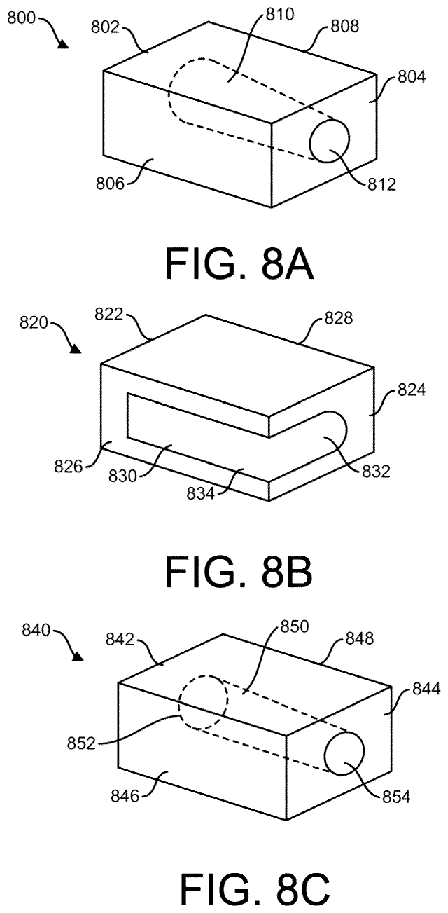

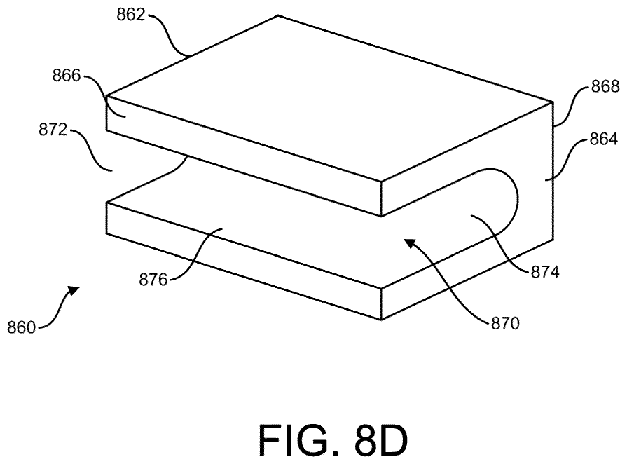

FIGS. 8A to 8D illustrate four of a plurality of general housing embodiments with particular guidewire passageways. The guidewire passageways can be characterized as one of open or closed and one of side loading and axial loading. The guidewire passageway configurations can be implemented in housing designs.

FIG. 8A illustrates a first embodiment of a housing 800 characterized as a closed and axial loading housing. The housing 800 include a proximal side 802, a distal side 804, and two longitudinal sides 806, 808. The housing 800 further includes a guidewire passageway 810 having an opening 812 on the distal side 804. The conductive member having the bladed member are disposed in the guidewire passageway 810. The guidewire passageway 810 does not include an opening on the on the proximal side 802 or on the longitudinal sides 806, 808. In this embodiment, the access to the guidewire passageway 810 is via the single opening 812. A proximal portion of the guidewire is loaded axially into the closed guidewire passageway 810. The conductive member penetrates the proximal portion of the shaft. In some embodiments, the closed and axial loading configuration of the housing 800 is implemented to penetrate at a precise location on the shaft.

FIG. 8B illustrates a second embodiment of a housing 820 characterized as a closed and side loading housing. The housing 820 include a proximal side 822, a distal side 824, and two longitudinal sides 826, 828. The housing 820 further includes a guidewire passageway 830 having a first opening 832 on the distal side 824 and a second opening 834 on longitudinal side 826. The first opening 832 can be one of a side opening or an axial opening and the second opening 834 can be the other of the side opening and the axial opening. The conductive member having the bladed member are disposed in the guidewire passageway 830. The guidewire passageway 830 in this embodiment does not include an opening on the on the proximal side 822. In this embodiment, the access to the guidewire passageway 810 is via the side and an axial opening, such as openings 832, 834. A proximal portion of the guidewire is loaded into the guidewire passageway 810 such as via the side opening, axial opening, or a combination of both the side and axial opening. The conductive member penetrates the proximal portion of the shaft. In some embodiments, the closed and side loading configuration of the housing 820 is implemented to penetrate at a precise location on the shaft.

FIG. 8C illustrates a third embodiment of a housing 868400 characterized as an open and axial loading housing. The housing 840 include a proximal side 842, a distal side 844, and two longitudinal sides 846, 848. The housing 840 further includes a guidewire passageway 850 having an opening 852 on the distal side 844 and an opening 854 on the proximal side 842. In this embodiment, the guidewire passageway 850 passed through housing 840. The conductive member having the bladed member are disposed in the guidewire passageway 850. The guidewire passageway 850 in this embodiment does not include an opening on the longitudinal sides 846, 848. In this embodiment, the access to the guidewire passageway 850 is via either opening 852, 854. The guidewire can be loaded axially into the open guidewire passageway 850 generally along the shaft rather than just at a proximal end. The open and axially loading embodiment of the housing 860 includes a conductive member implemented to penetrate the guidewire at a selected location on the shaft rather than just a proximal portion of the guidewire.

FIG. 8D illustrates a fourth embodiment of a housing 860 characterized as an open and side loading housing. The housing 860 include a proximal side 862, a distal side 864, and two longitudinal sides 866, 868. The housing 860 further includes a guidewire passageway 870 having a first opening 872 on the distal side 864, a second opening 874 on proximal side 864, and a third opening 896 on the longitudinal side 876. The first opening 892 is an axial opening, the second opening 894 is a side opening, and the second opening 864 is another axial opening opposite the first axial opening 892. The conductive member having the bladed member are disposed in the guidewire passageway 870. In this embodiment, the access to the guidewire passageway 870 is via the side opening 876 and the axial openings 872, 874. The guidewire is loaded into the guidewire passageway 870 such as via the side opening, axial opening, or a combination of both the side and axial opening. The open and side loading embodiment of the housing 860 includes a conductive member implemented to penetrate the guidewire at a selected location on the shaft rather than just a proximal portion of the guidewire.

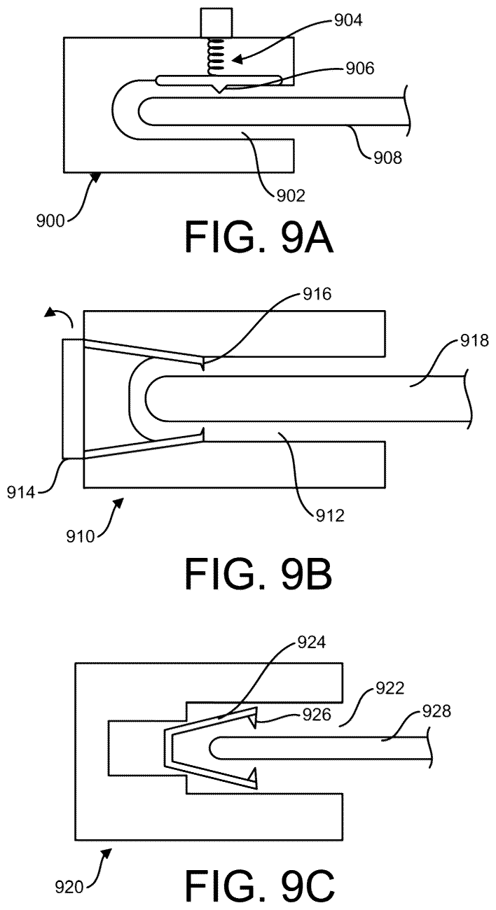

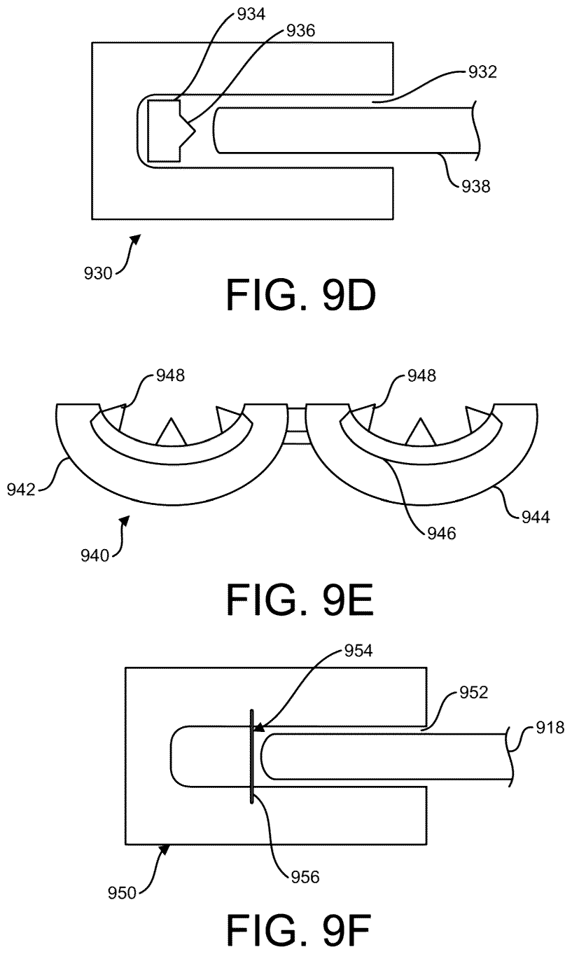

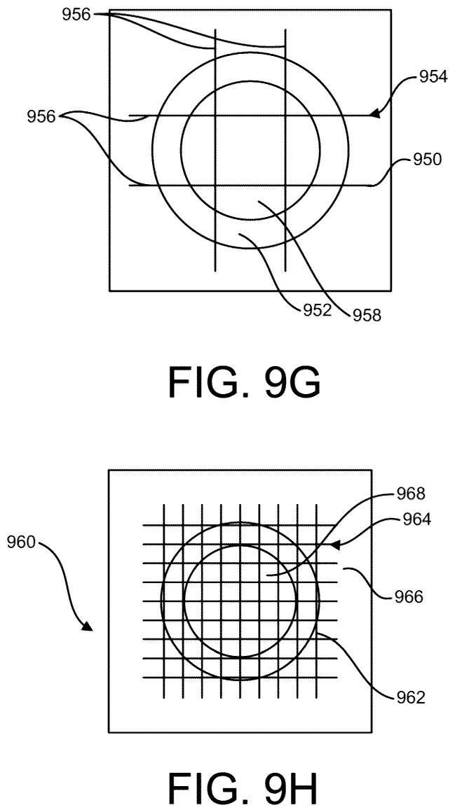

FIGS. 9A-9H illustrate eight of a plurality of general connection mechanisms of the conductive member in guidewire passageway of a housing to make mechanical and electrical contact with a mandrel through the electrical insulator of a guidewire. The connection mechanisms can be implemented with various embodiments of guidewire passageways and bladed members in housing designs. In some of the embodiments, insertion of the guidewire into the guidewire passageway facilitates penetration of the electrical insulator. In some of the embodiments, penetration is performed after the guidewire is inserted into the guidewire passageway. In some of the embodiments, the electrical insulator is destroyed in locations other than the penetration site for a guidewire removed from the guidewire passageway of the housing. In some embodiments, the electrical insulator is retained along the shaft other than at the penetration site for a guidewire removed from the guidewire passageway of the housing.

FIG. 9A illustrates a schematic view of housing 900 having a guidewire passageway 902 and a connection mechanism 904 including a bladed member 906 in which the bladed member penetrates a once loaded guidewire 908 with a lateral motion. For example, the guidewire 908 is inserted into the guidewire passageway 902, and then a button is pressed for the bladed member 906 to laterally penetrate the electrical insulator and make mechanical contact with the mandrel. The bladed member 902 is coupled to a conductive member in electrical communication with the cable. Other implementations of the connection mechanism 904, such as a vice or chuck, or pneumatic catcher can be applied. The connector mechanism can be implemented with generally any embodiment of the bladed member, with an open or closed, or axially loading or side loading guidewire passageway, penetration is performed after the guidewire is inserted into the guidewire passageway, and can be implemented to either destroy the electrical insulator or retain the electrical insulator other than the penetration site.

FIG. 9B illustrates a schematic view of housing 910 having a guidewire passageway 912 and a connection mechanism 914 including a bladed member 916 in which the bladed member 916 penetrates a once loaded guidewire 918 with a rotational motion. For example, the guidewire 918 is inserted into the guidewire passageway 912, and then a knob is turned for the bladed member 916 to rotationally penetrate the electrical insulator and make mechanical contact with the mandrel. The bladed member 912 is coupled to a conductive member in electrical communication with the cable. The connector mechanism 914 can be implemented with generally any embodiment of the bladed member, with a closed and axially loading or side loading guidewire passageway, penetration is performed after the guidewire 918 is inserted into the guidewire passageway 912, and can be implemented to either destroy the electrical insulator or retain the electrical insulator other than the penetration site.

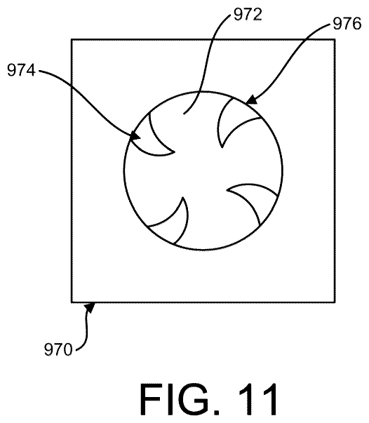

FIG. 11 illustrates an embodiment of housing 910 as housing 970 with guidewire passageway 972 and a rotatable connection mechanism 974 that includes a plurality of bladed members 976, which includes concave curved blades, such as bladed member 360 of FIG. 3D. The bladed member 976 can form a pupil that can dilate in a first configuration to open and allow a guidewire to enter or exit the connection mechanism 974 and constrict in a second configuration and hold the guidewire so as to penetrate the electrical insulator and make mechanical contact with the mandrel. Connection mechanism 970 can be implemented such that as a knob is turned in a first direction, the bladed member can rotate from the first configuration to the second configuration. As the knob is turned in an opposite direction, the bladed members 976 rotate from the second configuration to the first configuration.

FIG. 9C illustrates a schematic view of housing 920 having a guidewire passageway 922 and a connection mechanism 924 including a bladed member 926 in which the bladed member 926 penetrates a guidewire 928 with a clamping motion, such as a front-clamping motion, as the guidewire 928 is being loaded into the passageway 922. For example, the guidewire 928 is presses against the connection mechanism 924 as the guidewire 928 is inserted into the guidewire passageway 922. As the guidewire 928 presses against the connection member 924, the bladed members 926 close to penetrate the electrical insulator and make mechanical contact with the mandrel. The bladed member 926 is coupled to a conductive member in electrical communication with the cable. The connector mechanism 924 can be implemented with generally any embodiment of the bladed member, with a closed and axially loading or side loading guidewire passageway, penetration is performed while the guidewire is inserted into the guidewire passageway, and can be implemented to either destroy the electrical insulator or retain the electrical insulator other than the penetration site.

FIG. 9D illustrates a schematic view of housing 930 having a guidewire passageway 932 and a connection mechanism 934 including a bladed member 936 in which a stationary bladed member 936 penetrates a guidewire 938 as the guidewire 938 is being loaded into the passageway 932. For example, the guidewire 938 is presses against the bladed member 936 as the guidewire 938 is inserted into the guidewire passageway 932. As the guidewire 938 presses against the bladed member 936, the bladed member 936 penetrates the electrical insulator and makes mechanical contact with the mandrel. The bladed member 936 is coupled to a conductive member in electrical communication with the cable. The connector mechanism 934 can be implemented with generally any embodiment of the bladed member, with an open or closed and axially loading or side loading guidewire passageway, penetration is performed while the guidewire is inserted into the guidewire passageway, and can be implemented to either destroy the electrical insulator or retain the electrical insulator other than the penetration site.

FIG. 9E illustrates a schematic view of a housing 940 that can form a guidewire passageway as multiple sections, such as halves 942, 944, are brought together in a connection mechanism 946 in which one or more of the sections includes a bladed member 948. For example, a guidewire is placed against one of the sections and the connection mechanism is brought together such that the bladed member 948 is pressed against the guidewire. The bladed member 948 is coupled to a conductive member in electrical communication with the cable. The connector mechanism 946 can be implemented with generally any embodiment of the bladed member, with an open or closed and side loading guidewire passageway, penetration is performed after the guidewire is inserted into the housing 940, and can be implemented to either destroy the electrical insulator or retain the electrical insulator other than the penetration site.

FIG. 9F-9G illustrate schematic views of a housing 950 having a guidewire passageway 952 and a connection mechanism 954 including a bladed member 956 configured as a deflectable, sharp element, such as a wire strung across the guidewire passageway 952. A guidewire 958 is inserted into the guidewire passageway 952. FIG. 9F illustrates a side view of the housing 950 with the guidewire 958 inserted into the guidewire passageway 952, and FIG. 9G illustrates a rear view of the housing 950 with the guidewire 958 inserted into the guidewire passageway 952. The sharp element of the bladed member 956 slices the electrical insulator from the shaft to expose and mechanically contact the mandrel as the guidewire 956 is inserted into the guidewire passageway 952. The sharp element can be strung across the guidewire passageway 952 in a number of possible configurations. An axially inserted guidewire 958 is configured to pass by or through an element or a plurality of elements. In some embodiments, the elements are spaced apart from each other at a distance less than the diameter of the mandrel at the proximal portion of the shaft. The guidewire 958 can force aside an element or force apart the elements as the guidewire is inserted into the guidewire passageway 952, and the taut elements are urged against the shaft to slice the electrical insulator. As the guidewire 938 presses against the elements of the bladed member 956, the bladed member 956 penetrates the electrical insulator and makes mechanical contact with the mandrel. The elements are formed from a conductive material. The bladed member 956 is coupled to a conductive member in electrical communication with the cable. The connector mechanism 954 can be implemented with an open or closed and axially loading or side loading guidewire passageway, penetration is performed while the guidewire is inserted into the guidewire passageway, and generally destroys the electrical insulator on the proximal end.

FIG. 9H illustrates a housing 960 having a guidewire passageway 962 from a rear view of the guidewire passageway 962 and includes a connector 964 that is an alternative embodiment of the connector 954 in housing 950. For example, where the elements are spaced apart or configured to allow a guidewire to pass axially alongside the elements in housing 950, FIG. 9H illustrates a bladed member 966 having elements that are spaced apart at a close enough distance in which the elements do not deflect enough to allow a guidewire 968 to axial pass through the bladed member 966 in the guidewire passageway 962. For example, the sharp elements of the bladed member 966 form a screen that penetrates the proximal tip of the guidewire 968 but not the longitudinal sides of the guidewire 968. As the guidewire 968 presses against the elements of the bladed member 966, the bladed member 966 penetrates the electrical insulator and makes mechanical contact with the mandrel on the proximal tip of the guidewire 968. The elements are formed from a conductive material. The bladed member 966 is coupled to a conductive member in electrical communication with the cable. The connector mechanism 964 can be implemented with a closed and axially loading or side loading guidewire passageway, penetration is performed while the guidewire is inserted into the guidewire passageway, and generally does not destroy the electrical insulator on the longitudinal side of the proximal end.

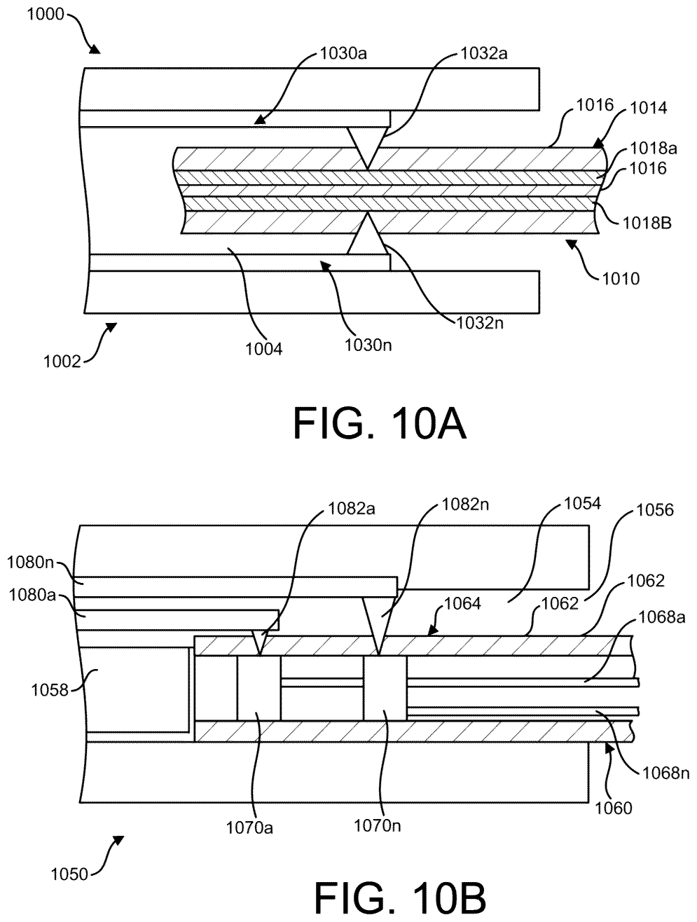

FIGS. 10A and 10B illustrate a feature of electrical connectors 1000, 1050 for use with an interventional assembly of the electrical connector couplable to a guidewire or the electrosurgical system 100. In some embodiments, a guidewire member of the assembly includes multiple electrodes arranged in an electrode assembly in which a plurality of electrodes receive or deliver separate electrical signals from other electrodes in the electrode assembly. In some embodiments, the electrode assembly includes an active electrode and a return electrode for configuration of the guidewire member in a bipolar configuration. The electrode assembly can include a plurality of spaced-apart electrodes or multiple spaced-apart sets or groups of spaced-apart electrodes on a distal portion of the guidewire member. Each of the plurality of electrodes is electrically coupled to a corresponding elongated mandrel that extends along the shaft to the proximal end of the guidewire. In one example, each electrode of the spaced-apart electrodes corresponds with a separate, single mandrel. In another example, a plurality of electrodes may be coupled to a mandrel. Other configurations are contemplated. The mandrels are insulated from one another within the shaft, such as with an insulating polymer sheath, and the entire proximal portion of the shaft is covered in an electrical insulator. Connectors 1000, 1050 illustrate two embodiments for use in an assembly having multiple electrode-mandrel guidewires.

FIG. 10A illustrates a schematic side view of a connector 1000 couplable to a multiple electrode-mandrel guidewire 1010, such as a guidewire having a plurality of mandrels radially spaced apart at the proximal end. In the illustrated embodiment, the guidewire 1010 includes a shaft 1014. The shaft 1014 includes an electrical insulator 1016 disposed on a plurality of mandrels 1018a . . . 1018n in which the electrical insulator 1016 covers the entire mandrel 1018 prior to the guidewire 1010 being inserted into the connector 1000. The mandrels 1018 are electrically isolated from each other within the shaft 1014, and each mandrel 1018a . . . 1018n is conductively coupled to an associated electrode exposed on the distal end of the guidewire 1010.

The connector 1000 includes a housing 1002 and a guidewire passageway 1004. In the illustrated embodiment, the guidewire 1010 is inserted axially into the guidewire passageway 1004. A plurality of conductive member 1030 are disposed in the guidewire passageway 1004. The conductive members 1030a . . . 1030n correspond with a mandrel 1018a . . . 1018n. Each conductive member 1030a . . . 1030n is electrically coupled to a corresponding lead conductor of the cable. The cable can include a proximal plug that includes a pin for each lead conductor to separately provide or deliver an electrical signal with the controller. A bladed member 1032a . . . 1032n is electrically and mechanically coupled to the associated conductive member 1030a . . . 1030n. The bladed members 1032a . . . 1032n are radially spaced apart and disposed within the guidewire passageway 1004 to penetrate the electrical insulator 10 and mechanically contact the associated mandrel 1018a . . . 1018n of the guidewire 1010. The guidewire 1010 is held in place by a friction fit between the bladed members 1032, which act as support members. The bladed members 1032 in mechanical contact with the associated mandrels 1018 provide an electrical connection between the mandrels 1018 and the lead conductors in the cable conductively coupled to the conductive members 1030. In some embodiments, the guidewire 1010 is particularly shaped on the shaft 1014 or includes visual indicators to fit within the guidewire passageway 1004 in a manner to properly couple the bladed members 1032 with the associated mandrels 1018. The connector 1000 can incorporate various clamp connectors and pushbuttons to lock and release or otherwise cause the bladed members 1032 to penetrate the electrical insulator 1016. With the multiple radially spaced apart mandrels 1018, the housing 1002 can be configured as an open or closed housing.

FIG. 10B illustrates a schematic side view of a connector 1050 couplable to a multiple electrode-mandrel guidewire 1060, such as a guidewire having a plurality of mandrels having connectors 1070 longitudinally spaced apart at the proximal end. In the illustrated embodiment, the guidewire 1060 includes a proximal portion 1062 of a shaft 1064. The proximal portion 1062 includes an electrical insulator 1066 disposed on a plurality of mandrels 1068a . . . 1068n in which the electrical insulator 1066 covers the entire mandrel proximal portion 1062 prior to the guidewire 1060 being inserted into the guidewire passageway of the connector 1050. The mandrels 1068 are electrically isolated from each other within the shaft 1064, and each mandrel 1068a . . . 1068n is conductively coupled to an associated electrode exposed on the distal end of the guidewire 1060. Further, each mandrel 1068a . . . 1068n is conductively coupled to an associate connector 1070a . . . 1070n longitudinally spaced-apart from each other on the proximal portion 1062 of the guidewire 1060. The connectors 1070 can extend radially around the shaft 1064, such as a ring conductor, disposed underneath the electrical insulator 1066 prior to the guidewire 1010 being inserted into the connector 1050.

The connector 1050 includes a housing 1052 and a guidewire passageway 1054. In the illustrated embodiment, the guidewire 1060 is inserted axially into the guidewire passageway 1054. The guidewire passageway 1054 can include an opening 1056 at one end and a stopper 1058 at the opposite end. A plurality of conductive member 1080 are disposed in the guidewire passageway 1054. The conductive members 1080a . . . 1080n correspond with a mandrel 1068a . . . 1068n. Each conductive member 1080a . . . 1080n is electrically coupled to a corresponding lead conductor of the cable. The cable can include a proximal plug that includes a pin for each lead conductor to separately provide or deliver an electrical signal with the controller. A bladed member 1082a . . . 1082n is electrically and mechanically coupled to the associated conductive member 1080a . . . 1080n. The bladed members 1082a . . . 1082n are longitudinally spaced apart and disposed within the guidewire passageway 1054 to penetrate the electrical insulator 1066 and mechanically contact the associated radial connectors 1070a . . . 1070n corresponding with mandrels 1068a . . . 1068n of the guidewire 1060. In some embodiments, the bladed members are radially spaced apart as well as longitudinally spaced apart. In the illustrated embodiment, the guidewire 1060 is inserted into the guidewire passageway 1054 until a proximal tip 1072 is in contact with the stopper 1058 to properly align the bladed members 1082 with the associated radial connectors 1070. The guidewire 1060 is held in place by a friction fit between the bladed members 1082, which act as support members. The bladed members 1082 in mechanical contact with the associated mandrels 1068 provide an electrical connection between the mandrels 1068 and the lead conductors in the cable conductively coupled to the conductive members 1080. The connector 1050 can incorporate various clamp connectors and pushbuttons to lock and release or otherwise cause the bladed members 1082 to penetrate the electrical insulator 1066

Various modifications and additions can be made to the exemplary embodiments discussed without departing from the scope of the present disclosure. For example, while the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

We claim:

1. An electrosurgical assembly for use with an electrosurgical controller, the electrosurgical assembly comprising:
a guidewire having an electrically conductive mandrel extending along a shaft from a proximal end to a distal portion, the distal portion having an electrode electrically coupled to the mandrel, the shaft having an electrical insulator disposed on the mandrel and extending to a proximal end; and
a connector configured to releasably couple to the guidewire, the connector comprising;
a cable configured to be coupled to the electrosurgical controller,
a housing coupled to the cable, the housing having a guidewire passageway configured to receive the shaft of the guidewire, and
a conductive member disposed within the housing and electrically coupled to the cable, the conductive member having a conductive contact member, the conductive contact member adjacent the guidewire passageway and configured to penetrate the electrical insulator and make electrical contact with the mandrel when the shaft of the guidewire is disposed in the guidewire passageway.

2. The electrosurgical assembly of claim 1, wherein the housing includes a clamp connector configured to releasably hold the shaft of the guidewire, the clamp connector including a support member to urge the shaft against the contact member.

3. The electrosurgical assembly of claim 2, wherein the contact member is a first contact member, and the support member includes a second contact member electrically coupled to the electrical connector.

4. The electrosurgical assembly of claim 2, wherein the clamp connector includes a spring coupled to the housing, wherein the spring is configured to urge the shaft against the contact member.

5. The electrosurgical assembly of claim 2, wherein the housing further includes a release button mechanically coupled to the clamp connector, the release button transitionable from a first position to a second position with respect to the housing, wherein the transition from the first position to the second position releases guidewire from the clamp connector.

6. The electrosurgical assembly of claim 1, wherein the contact member includes one of a tapered edge configured to cut the electrical insulator and a point configured to pierce the electrical insulator and make mechanical contact with the mandrel.

7. The electrosurgical assembly of claim 6, wherein the contact member including the edge includes one of a straight edge and a serrated edge.

8. The electrosurgical assembly of claim 1, wherein the guidewire includes a plurality of electrodes.

9. The electrosurgical assembly of claim 8, wherein the connector includes a plurality of spaced-apart conductive members, and the cable includes a plurality of lead conductors, where each conductive member is electrically coupled to a corresponding lead conductor of the plurality of lead conductors, and wherein the guidewire includes a plurality of mandrels, each mandrel is coupled to a corresponding electrode of the plurality of electrodes, wherein each conductive member is couplable to a corresponding mandrel of the plurality of mandrels.

10. The electrosurgical assembly of claim 8, further comprising a plurality of ring connectors, wherein each mandrel is electrically coupled to a corresponding ring connector, and the ring connectors are longitudinally spaced-apart at a proximal end of the shaft.

11. The electrosurgical assembly of claim 10, wherein the conductive members are radially spaced apart.

12. The electrosurgical assembly of claim 1, wherein the guidewire includes proximal portion of the shaft, and the housing is configured to receive the proximal portion of the shaft.

13. The electrosurgical assembly of claim 1, wherein the guidewire passageway includes one of a single opening and a plurality of openings for the guidewire passageway.

14. An electrosurgical system, comprising:
an electrosurgical controller;
a guidewire having an electrically conductive mandrel extending along a shaft from a proximal end to a distal portion, the distal portion having an electrode electrically coupled to the mandrel, the shaft having an electrical insulator disposed on the mandrel and extending to a proximal end, the guidewire electrically coupled to the electrosurgical controller; and
a connector configured to releasably couple to the guidewire, the connector comprising;
a cable configured to be coupled to the electrosurgical controller,
a housing coupled to the cable, the housing having a guidewire passageway configured to receive the shaft of the guidewire, and
a conductive member disposed within the housing and electrically coupled to the cable, the conductive member having a conductive bladed member, the conductive bladed member adjacent the guidewire passageway and configured to penetrate the electrical insulator and make mechanical and electrical contact with the mandrel when the shaft of the guidewire is disposed in the guidewire passageway.

15. The electrosurgical system of claim 14, wherein the electrosurgical controller is one of a radiofrequency (RF) generator and an electroanatomical mapping (EAM) controller.

16. The electrosurgical system of claim 15, wherein the electrosurgical controller is the RF generator, and the electrode is configured to operate in one of a monopolar mode and a bipolar mode.

17. The electrosurgical system of claim 15, wherein the electrosurgical controller is the EAM system, and the guidewire includes a plurality of electrodes on the distal portion of the shaft.

18. An electrosurgical connector for use with a guidewire having an electrically conductive mandrel extending along a shaft from a proximal end to a distal portion, the distal portion having an electrode electrically coupled to the mandrel, the shaft having an electrical insulator disposed on the mandrel and extending to a proximal end and an electrosurgical controller, the electrosurgical connector comprising:
a cable configured to be coupled to the electrosurgical controller;
a housing coupled to the cable, the housing having a guidewire passageway configured to receive the shaft of the guidewire and releasably couple the connector to the guidewire; and
a conductive member disposed within the housing and electrically coupled to the cable, the conductive member having a conductive contact member, the conductive contact member adjacent the guidewire passageway and configured to penetrate the electrical insulator and make electrical contact with the mandrel when the shaft of the guidewire is disposed in the guidewire passageway.

19. The electrosurgical connector of claim 18, wherein the conductor contact member includes a plurality of conductive bladed members and wherein the housing includes a clamp connector configured to releasably hold the shaft of the guidewire, the clamp connector to urge the shaft against the plurality of bladed members.

20. The electrosurgical connector of claim 19, wherein the clamp connector includes a spring coupled to the housing, wherein the spring is configured to urge the shaft against the bladed members.
